(19) United States
(12) Patent Application Publication
(10) Pub. No.: US 2009/0036759 A1
(43) Pub. Date: Feb. 5, 2009

(54) COLLAPSIBLE NONINVASIVE ANALYZER METHOD AND APPARATUS

(76) Inventors: Timothy E. Ault, Chandler, AZ (US); Stephen L. Monfre, Gilbert, AZ (US); Kevin H. Hazen, Gilbert, AZ (US)

Correspondence Address: GLENN PATENT GROUP, 3475 EDISON WAY, SUITE L, MENLO PARK, CA, 94025, US

(21) Appl. No.: 12/179,495

(22) Filed: Jul. 24, 2008

Related U.S. Application Data
(60) Provisional application No. 60/953,448, filed on Aug. 1, 2007.

(51) Int. Cl. A61B 5/1455

(52) U.S. Cl. 600/310

(57) ABSTRACT

The invention relates generally to a noninvasive spectroscopic based analyzer. More particularly, a collapsible spectrometer and or deployable subject interface for an analyzer, such as a noninvasive glucose concentration analyzer, is described.

### CROSS-REFERENCE TO RELATED APPLICATION

This application claims benefit of U.S. provisional patent application Ser. No. 60/953,448 filed Aug. 1, 2007, which application is incorporated herein in its entirety by this reference thereto.

### BACKGROUND OF THE INVENTION

1. Field of the Invention

The invention relates generally to spectroscopic measurement of analyte properties in tissue. More particularly the invention relates to a collapsible spectrometer for noninvasive measurements. In one embodiment, near-infrared measurement of glucose concentration in tissue is performed using a partially collapsible near-infrared analyzer.

2. Discussion of the Related Art

### Noninvasive Technologies

There are a number of reports on noninvasive technologies. Some of these relate to general instrumentation configurations, such as those required for noninvasive glucose concentration estimation, while others refer to sampling technologies. Those related to the present invention are briefly reviewed here:

P. Rolfe, Investigating substances in a patient's bloodstream, U.K. patent application Ser. No. 2,033,575 (Aug. 24, 1979) describes an apparatus for directing light into the body, detecting attenuated backscattered light, and using directing light into the body, detecting attenuated backscattered light, and using the collected signal to determine glucose concentrations in or near the bloodstream.

C. Dahne, D. Gross, Spectrophotometric method and apparatus for the non-invasive, U.S. Pat. No. 4,655,225 (Apr. 7, 1987) describe a method and apparatus for directing light into a patient's body, collecting transmitted or backscattered light, and determining glucose concentrations from selected near-infrared wavelength bands. Wavelengths include 1560 to 1590, 1750 to 1780, 2085 to 2115, and 2255 to 2285 nm with at least one additional reference signal from 1000 to 2700 nm.

R. Barnes, J. Brasch, D. Purdy, W. Lougheed, Non-invasive determination of analyte concentration in body of mammals, U.S. Pat. No. 5,379,764 (Jan. 10, 1995) describe a noninvasive glucose concentration estimation analyzer that uses data pretreatment in conjunction with a multivariate analysis to estimate blood glucose concentrations.

M. Robinson, K. Ward, R. Eaton, D. Haaland, Method and apparatus for determining the similarity of a biological analyte from a model constructed from known biological fluids, U.S. Pat. No. 4,975,581 (Dec. 4, 1990) describe a method and apparatus for measuring a concentration of a biological analyte, such as glucose concentration, using infrared spectroscopy in conjunction with a multivariate model. The multivariate model is constructed from a plurality of known biological fluid samples.

J. Hall, T. Cadell, Method and device for measuring concentration levels of blood constituents non-invasively, U.S. Pat. No. 5,361,758 (Nov. 8, 1994) describe a noninvasive device and method for determining analyte concentrations within a living subject using polychromatic light, a wavelength separation device, and an array detector. The apparatus uses a receptor shaped to accept a fingertip with means for blocking extraneous light.

K. Hazen, G. Acosta, N. Abul-Haj, and R. Abul-Haj Apparatus and method for reproducibly modifying localized absorption and scattering Coefficients at a tissue measurement site during optical sampling, U.S. Pat. No. 6,534,012 (Mar. 18, 2003) describe a noninvasive glucose concentration analyzer having a hand and elbow stabilizer for use during noninvasive glucose concentration determination.

As seen in these references, a noninvasive analyzer includes a number of elements, such as: a source, backreflector, incident light directing optics, a subject interface module, light collecting optics, a detector, temperature controller, coupling fluid delivery components, processor, and display. Further, the subject interface module often includes a number of elements, such as positioning elements for various body parts. Many of the analyzer components are sensitive to shock, electric fields, water, temperature, and/or dust. Combined, the analyzer includes a large number of elements that must be protected from the environment. This results in a bulky analyzer that is hard to transport, is fragile, and takes up a lot of space.

Clearly, there exists a need for a spectroscopic analyzer and subject interface that is still portable, readily used, and adjustable to fit a large range of sample sizes.

### SUMMARY OF THE INVENTION

The invention relates generally to a noninvasive spectroscopic based analyzer. More particularly, a collapsible spectrometer and/or deployable subject interface for an analyzer, such as a noninvasive glucose concentration analyzer, is described.

### BRIEF DESCRIPTION OF THE DRAWINGS

FIG. 1 illustrates an analyzer interfacing with a human body;

FIG. 2 illustrates a noninvasive analyzer including a base module, a communication bundle, and a sample module that is controlled by an algorithm;

FIGS. 3A and 3B illustrate a noninvasive analyzer in (FIG. 3A) a closed configuration and (FIG. 3B) in an open configuration;

FIG. 4 illustrates a deployable subject interface module;

FIG. 5 illustrates an analyzer having a transformable subject interface module;

FIG. 6 illustrates a transformable analyzer computer combination;

FIG. 7 illustrates an analyzer in a carrying case;

FIG. 8 illustrates pop-out arm interface; and

FIG. 9 illustrates a controller/actuator controlled sample probe.

### DETAILED DESCRIPTION OF THE INVENTION

The invention comprises a noninvasive analyzer that stores or transports in a compact format and operates in an expanded, transformed, or unfolded state. Generally, the analyzer is referred to as any of collapsible, deployable, or transformable.

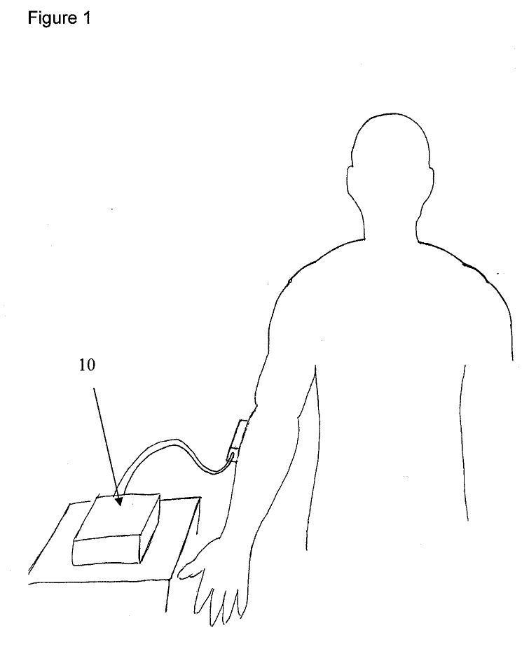

Referring now to FIG. 1, an analyzer is illustrated interfacing with a human body. The analyzer, described supra, interfaces with any skin surface of the human body.

### Instrumentation

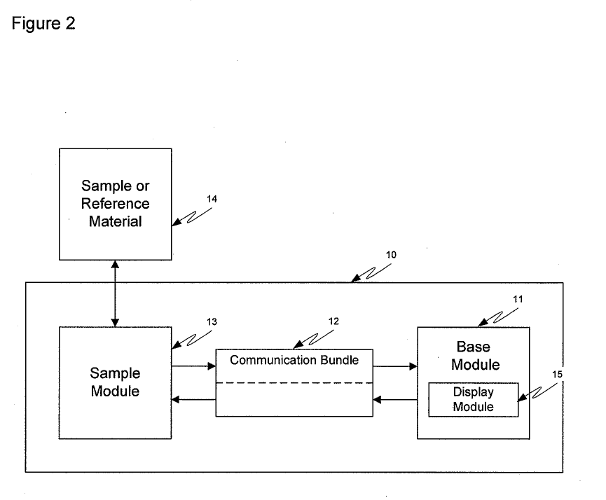

Referring now to FIG. 2, a noninvasive analyzer is illustrated. The analyzer 10 includes at least a source, illumination optics, collection optics, a detector, and an analysis algorithm. The analyzer 10 optionally includes a base module 11, communication bundle 12, and sample module 13. The base module has a display module. The analyzer components are optionally separated into separate housing units or are integrated into a single unit, such as a handheld unit. Preferably, a source is integrated into either the base module or the sample module. In a first case, the source element is integrated into the base module and the communication bundle carries the incident optical energy to the sample. In a second preferred case, the source element is integrated into the sample module. In both cases, photons are directed toward the tissue sample via a sample probe that is part of the sample module and the photonic signal collected from the sample by the sampling module is carried to a detector, typically in the base module, via the communication bundle.

Preferably, a signal processing means results in a control signal that is transferred from the base module via the communication bundle back to the sampling module. The communicated control signal is used to control the movement, such a position and attitude of the sample probe relative to the tissue sample or reference material.

### Analyzer Transformation

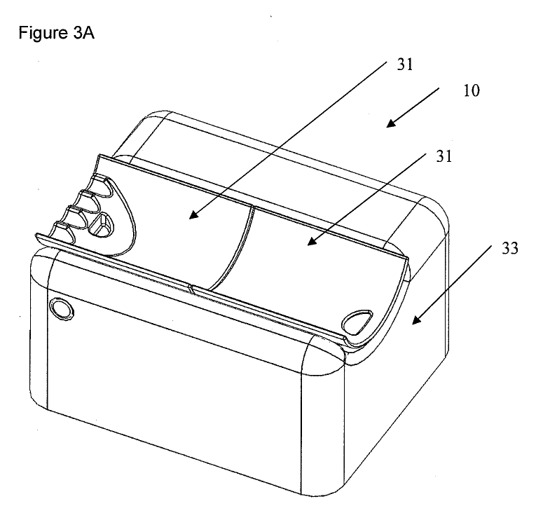

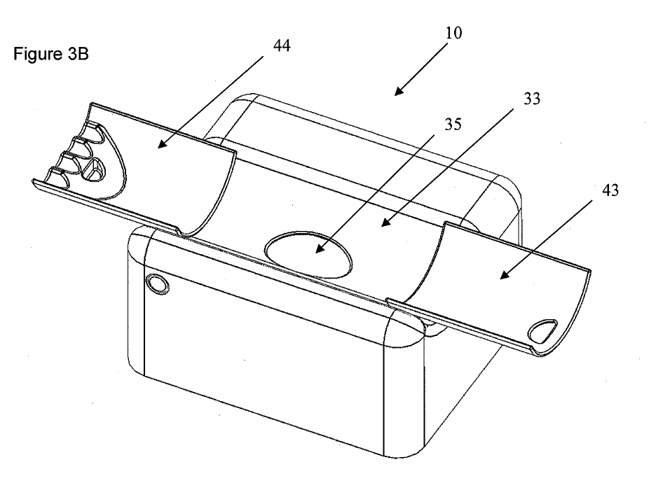

In one embodiment, an analyzer is transported and/or stored in a closed or folded state and operated in an open or unfolded state. Referring now to FIGS. 3A and 3B, an example of an analyzer 10 in a closed state FIG. 3A and open state FIG. 3B is illustrated. In this example, the analyzer is opened to allow a subject to insert a portion of their body, such as a forearm, into the analyzer for analysis. When opening and closing, a top portion of the analyzer 31 moves relative to a bottom portion of the analyzer 33 along one or more support/guide rails. Opening the analyzer optionally exposes an optical interface between the subject and a testing site 35. Preferably this motion is automated and under algorithm control. The ability to close the analyzer when not in use has a number of benefits including:
protection of optics from physical damage;
protection of sensitive analyzer components from contamination; and
ease of transport.

The ability to place the sample site into the analyzer has a number of benefits including:
an optical train with optionally fixed relative location of optical components, which minimizes optical noise and wear from movement of optics; and
a reduced footprint of the analyzer.

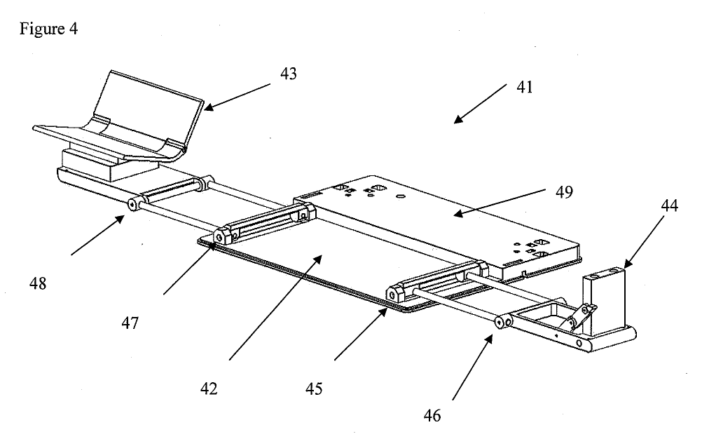

Once open, the analyzer optionally has a subject interface that mechanically adjusts to accommodate the sample. In this example, a wrist and/or hand rest 44 and an elbow rest 43 slide out to support a subject's arm. Manners in which the supports for the arm expand from the analyzer are further described along with the description of FIG. 4. Once a subject's arm is positioned inside the analyzer, the tip of the sample probe is positioned relative to a sample site. For instance, the tip of the sample probe is brought into proximate contact with the sample site of the subject's arm. Movement of the sample probe is achieved by moving the top of the analyzer relative to the sample site, or by adjusting the position and/or attitude of the sample probe tip. Descriptions of movement of the sample probe tip relative to the skin in terms of control, axis or movement, and degree of contact between the sample probe tip and sample site are described in:
U.S. patent application Ser. No. 11/117,104, filed Apr. 27, 2005;
U.S. patent application Ser. No. 11/625,752, filed Jan. 22, 2007; and
U.S. provisional patent No. 60/943,495 filed Jun. 12, 2007,
   which are all incorporated herein in their entirety by this reference thereto. In this example, the analyzer case contains a handle, grip, or hand slot 35 for ease of transport. Optionally, the lid of the analyzer flips open to reveal a display screen 51. Optionally, a coupling fluid reservoir is maintained inside of the analyzer, the coupling fluid is delivered through the sample probe tip, the coupling fluid is brought into the range of about 90 to 92 degrees Fahrenheit prior to delivery to the sample site, and/or delivery of the coupling fluid is performed in an automated process under algorithm control.

In another embodiment, a portion of the analyzer unfolds, extends, or expands prior to use. In this manner, the folded, unextended, or unexpanded portion of the analyzer takes up less space, is more readily transported, and is protected when not in use. The folded, extended, or expanded state of the analyzer facilitates a measurement process using the analyzer. The expansion of the portion of the analyzer is optionally automated and/or under computer control.

Referring now to FIG. 4, an example of a collapsible or foldable subject interface support 41 that is attached or replaceable attached to an analyzer is illustrated. In this case, the subject interface support is bolted to the analyzer through an analyzer interfacing plate 49. The interfacing plate can unfold from the analyzer, be replaceably attached to the analyzer, or slide out from the analyzer. In this example, an arm or elbow support 43 and a hand or finger support 44 are hingedly attached to a base support 42. The elbow support unfolds along a first axis 45 and a second axis 46 from a storage volume in the base support. A hand or elbow rest either pivots up from an extending portion or is replaceably attached to the extending portion of the hand support. Similarly, an elbow support unfolds along a third axis 47 and fourth axis 48. The elbow interfacing support is either integrated with the extending portion of the elbow support mechanism or is replaceably attached to the extending portion of the elbow support mechanism.

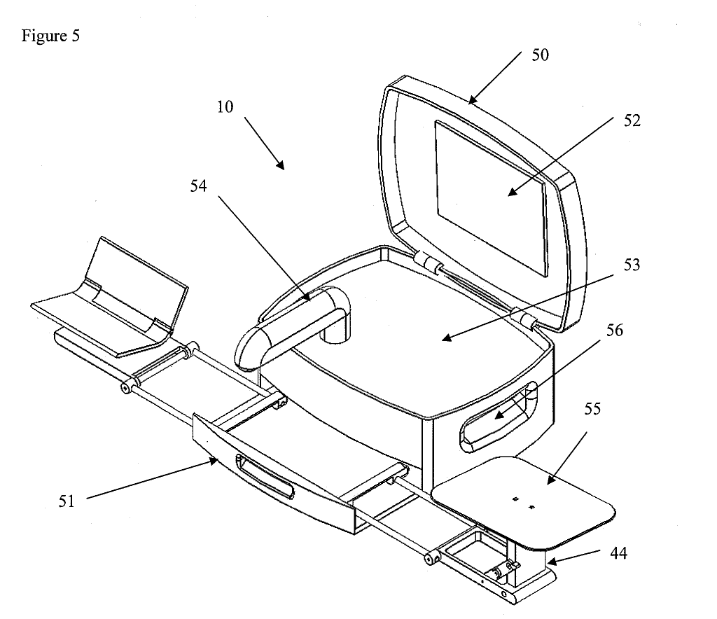

Referring now to FIG. 5, another embodiment of a collapsible analyzer is illustrated. In this example, an analyzer 10 having a pullout tray 51 that unfolds to form a subject interface 41 is illustrated. In this example, the analyzer contains a tray that slides from a closed position to an open position. Typically, the tray is maintained in a closed position while the analyzer is in a state of transport or storage. Prior to use the tray is configured to a deployed position through manual force or via automated software control. As illustrated in FIG. 4, the body part support elements are subsequently unfolded from the tray. In the illustrated case, a removably replaceable hand support is attached to the hand support element 44. In this case, the human body part support elements, such as a hand and elbow support either further unfold or deploy from the hinged elements or are parts replaceably attached to the analyzer. FIG. 5 further illustrates an analyzer having a lid that when opened reveals a display monitor 52 and user input controls 53, such as keyboard or touch screen input. Optionally, opening the lid of the analyzer reveals a sample probe 54 that is extendable or rotatable from the analyzer for subsequent data collection. Also illustrated in FIG. 5 is an indented hand hold 56 for facilitating transport of the analyzer.

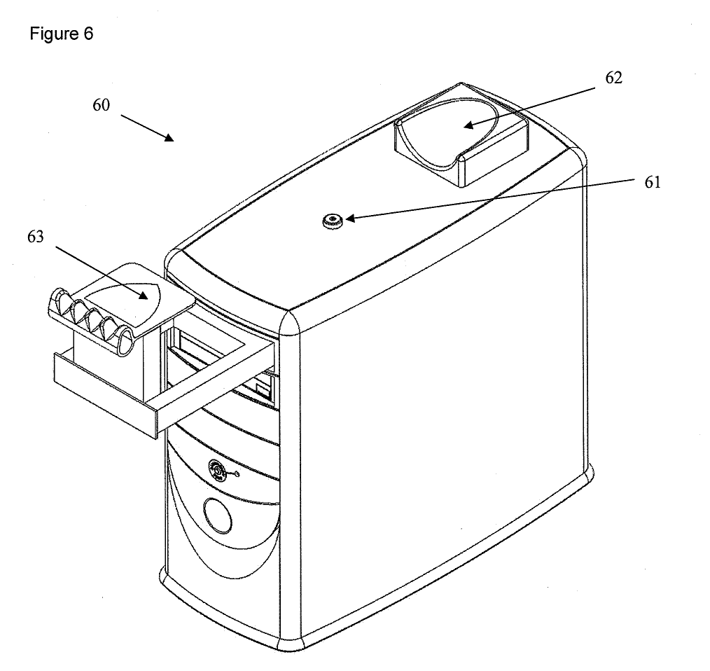

In still yet another embodiment of the invention, the analyzer is integrated into a personal computer. For example, a laptop or desktop personal computer contains the analyzer source, optics, sample interface, and detector. The personal computer supplies the processor, memory, display screen, and user input and output elements of the analyzer. In this manner, the analyzer also operates as a personal computer. This reduces the effective cost of the analyzer to the user. A first example of a laptop personal computer with added analyzer components is illustrated in FIG. 5. Referring now to FIG. 6, a second example of a noninvasive analyzer embedded into a tower configured personal computer 60 is illustrated. In this example, the spectrometer optical components are housed inside the personal computer tower case. A tip of a sample probe 61 extends from the tower case. The sample probe tip interfaces with a body part, such as an underside of a forearm, during use. In this example, an elbow support 62 is illustrated on the tower case top and a hand interface 63 is stored inside the case. The hand interface ejects like a compact disc from the tower and then folds upward into a position that combined with the elbow support aligns the arm over the sample probe tip for subsequent optical sampling.

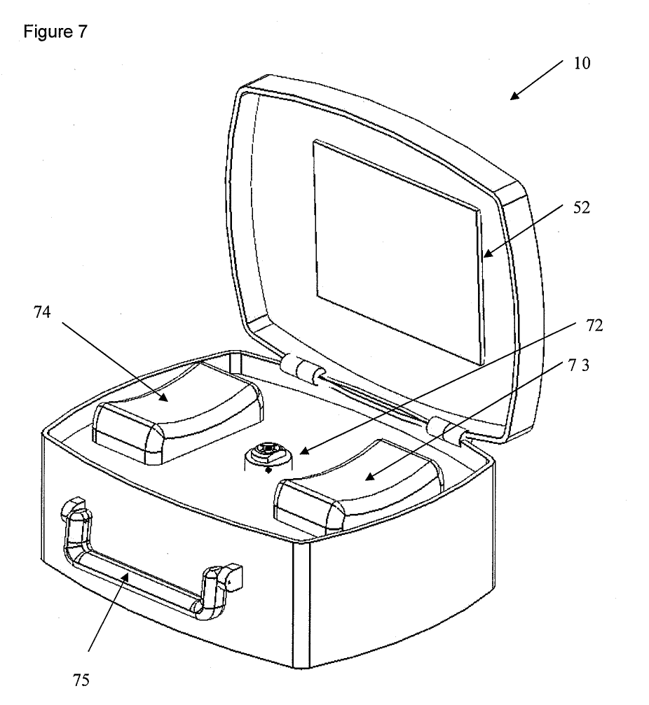

In yet another embodiment, an analyzer opens up or unfolds. The analyzer is transported and/or stored in a closed or folded state and operated in an open or unfolded state. Referring now to FIG. 7, an example of an analyzer 10 contained in a carrying case is presented. In this example, the case is hinged and contains inside the sealed environment a display screen 52, a sample probe head 72 of the sample module 13 and supports for the subject. Examples of supports include a wrist rest 73 and an elbow rest 74. In this example, the analyzer case contains a handle, grip, or hand slot 75 for ease of transport. The case preferably encloses the sensitive analyzer components so as to protect them from contamination and from physical damage during transport. Optionally, the analyzer unfolds to include a human interface, such as a keyboard, mouse, or other interactive computer input device.

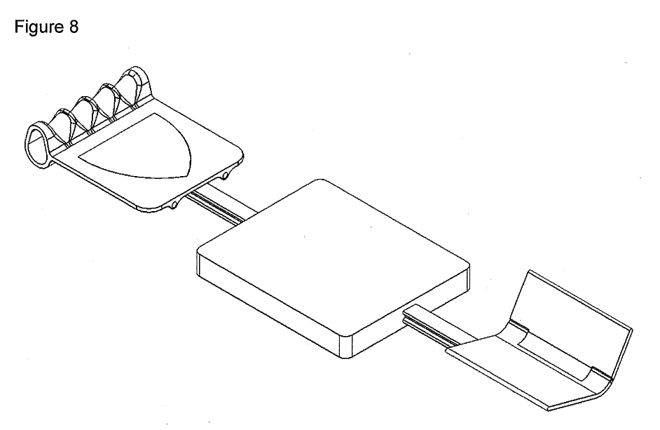

In yet another embodiment of the invention, a subject interface slides out from an enclosure of the analyzer. Referring now to FIG. 8, hand and elbow support deploy to an operating configuration along one or more rails. Preferably the rails slide on bearings and have a positive stop with a lock, such as a spring-loaded pin or clamp, to hold the supports in their deployed position.

The examples above illustrate particular cases of an analyzer or subject interface that expands or reconfigures for use. In these examples, slides and hinges are used to extend the subject interface portion of the analyzer. However, the inventors recognize that many mechanical system exist for expanding the analyzer or a portion of the analyzer. For example, the assembly may expand along a linear or nonlinear slide, use a spring and a catch, or pneumatically reposition. Generally, the expansion or reconfiguration is performed using any mechanical, pneumatic, and or electrical means in an automated or manual process. Similarly, terms such as unfolding or extending are used to describe the analyzer or analyzer portion transformation. However, the inventors recognize that many terms are usable to describe the process such as expansion, extension, transformation, or reconfiguration. Hence, the inventors conceive a transformation of at least a portion of the analyzer where the transformation is achieved using mechanical, pneumatic, and or electrical means in an automated or manual process to result in a collapsed state of analyzer taking up less room, protecting components, and/or facilitating transport and an expanded state that facilitates use of the analyzer.

### Coordinate System

Herein, positioning and attitude are defined. Positioning is defined using a x-, y-, and z-axes coordinate system relative to a given body part. A relative x-, y-, z-axes coordinate system is used to define a sample probe position relative to a sample site. The x-axis is defined along the length of a body part and the y-axis is defined across the body part. As an illustrative example using a sample site on the forearm, the x-axis runs between the elbow and the wrist and the y-axis runs across the axis of the forearm. Similarly, for a sample site on a digit of the hand, the x-axis runs between the base and tip of the digit and the y-axis runs across the digit. The z-axis is aligned with gravity and is perpendicular to the plane defined by the x- and y-axis. Further, the orientation of the sample probe relative to the sample site is defined in terms of attitude. Attitude is the state of roll, yaw, and pitch. Roll is rotation of a plane about the x-axis, pitch is rotation of a plane about the y-axis, and yaw is the rotation of a plane about the z-axis. Tilt is used to describe both roll and pitch.

### Tissue Stress/Strain

The controller optionally moves the sample probe so as to make minimal and/or controlled contact with a sample to control stress and/or strain on the tissue, which is often detrimental to a noninvasive analyte property determination. Strain is the elongation of material under load. Stress is a force that produces strain on a physical body. Strain is the deformation of a physical body under the action of applied force. In order for an elongated material to have strain there must be resistance to stretching. For example, an elongated spring has strain characterized by percent elongation, such as percent increase in length.

### Actuator/Controller

A controller controls the movement of one or more sample probes of the targeting and/or measuring system via one or more actuators. An actuator moves the sample probe relative to the tissue sample. One or more actuators are used to control the position and/or attitude of the sample probe. The actuators preferably acquire feedback control signals from the measurement site or analyzer. The controller optionally uses an intelligent system for locating the sample site and/or for determining surface morphology. Controlled elements include any of the x-, y-, and z-axes positions of sampling along with pitch, yaw, and/or roll of the sample probe. Preferably, a tip of a sample probe head of a sample module is controlled by an algorithm along a normal-to-skin-axis. Preferably, the sample probe head is positioned in terms of 3-D location in the x-, y-, and z-axes and is attitude orientated in terms of pitch, yaw, and roll. Further, attitude of the probe head is preferably orientated prior to contact of the sample probe head with the tissue sample using remote indicators, such as feedback from capacitance, optical, or electrical sensors. Also optionally controlled are periods of light launch, intensity of light launch, depth of focus, and surface temperature. Several examples signal generation used with the controller and actuator follow.

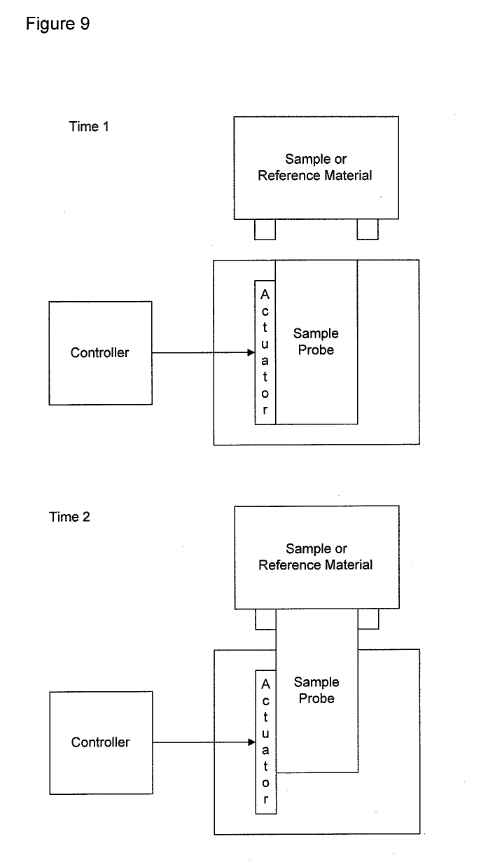

A schematic presentation of the sample module is presented in FIG. 9. The sample module includes an actuator and a sample probe. The actuator is driven by a controller. The controller sends the control signal from the algorithm to the sample module actuator via a communication bundle. The actuator subsequently moves the sample probe relative to the tissue sample site. The sample probe is controlled along the z-axis from a position of no contact, to a position of tissue sample contact, and optionally to a position of minimal tissue sample displacement. The sample probe is presented in FIG. 9 at a first and second period of time with the first time period presenting the sample probe when it is not in contact with the sample site. The second time period presents the sample probe with minimal displacement of the sample tissue.

In the foregoing discussion, the preferred embodiment of the invention is for the determination of a glucose concentration. Additional analytes for concentration or threshold determination are those found in the body including: water, protein, fat and/or lipids, blood urea nitrogen (BUN), both therapeutic and illicit drugs, and alcohol.

Although the invention has been described herein with reference to certain preferred embodiments, one skilled in the art will readily appreciate that other applications may be substituted without departing from the spirit and scope of the present invention. Accordingly, the invention should only be limited by the Claims included below.

1. An apparatus interfacing to a human subject, comprising:
a portable spectroscopic analyzer, said analyzer comprising:
means for deploying a tactile subject interface integrated into said analyzer, wherein said subject interface transforms from a non-operational configuration to an operational configuration.

2. The apparatus of claim 1, wherein said means for deploying said subject interface comprises an automated actuator controlled deployment of said subject interface.

3. The apparatus of claim 1, wherein said means for deploying said subject interface comprises any of:
a hinged mechanism used in deployment of said subject interface;
a rail mechanism used in deployment of said subject interface; and
a pneumatic system used in deployment of said subject interface.

4. The apparatus of claim 1, wherein said means for deploying said subject interface comprises any of:
unfolding said subject interface into said operational configuration from said non-operational configuration; and
extending said subject interface into said operational configuration from said non-operational configuration.

5. The apparatus of claim 4, wherein said non-operational configuration comprises any of:
a storage configuration; and
a transport configuration.

6. The apparatus of claim 1, wherein said means for deploying deploys any of:
a hand support;
a wrist support; and
an optical sample probe.

7. The apparatus of claim 1, further comprising a display screen, wherein said display screen becomes viewable to the human subject in said operational configuration and is not viewable to the human subject in said non-operational configuration.

8. The apparatus of claim 1, wherein said portable spectroscopic analyzer in said operational configuration exposes an optical sample probe interface upon deployment of said subject interface.

9. The apparatus of claim 1, further comprising a handle integrated into said portable spectroscopic analyzer.

10. The apparatus of claim 1, wherein said means for deploying combines unfolding at least a portion of said subject interface in combination with sliding said subject interface in transformation from said non-operational configuration to said operational configuration.

11. The apparatus of claim 1, wherein said means for deploying unfolds at least a first portion of said subject interface using a first hinge and unfolds a second portion of said first portion of said subject interface using a second hinge.

12. The apparatus of claim 1, wherein said means for deploying utilizes a slide out tray in transformation of said spectroscopic analyzer from said non-operational configuration to said operational configuration.
